(19) United States
(12) Patent Application Publication
(10) Pub. No.: US 2003/0235551 A1
(43) Pub. Date: Dec. 25, 2003

(54) Solubilized formulations containing nylon and suitable for personal care products and processes for the preparation thereof

(76) Inventor: Rolando U. Pagilagan, Parkersburg, WV (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/437,631

(22) Filed: May 14, 2003

Related U.S. Application Data
(60) Provisional application No. 60/380,486, filed on May 14, 2002.

(51) Int. Cl. A61K 7/06; A61K 7/11

(52) U.S. Cl. 424/70.17

(57) ABSTRACT

There is disclosed personal care products made from solubilized formulations containing nylons. These products can be applied in water-based solutions by using these polyamides and their copolymers, while retaining their traditional functions (as in shampoos and conditioners, deodorants, and makeup applications). Processes for their preparation are also disclosed, including forming the polyamide and adding the substrate material of interest.

### CROSS REFERENCE TO RELATED APPLICATIONS

This application claims the benefit of U.S. Provisional Application No. 60/380,486, filed May 14, 2002.

### FIELD OF THE INVENTION

This invention relates to the incorporation of water-soluble and alcohol-soluble nylons in products pertaining to personal care applications. More particularly, this invention relates to the manufacture and use of such materials for any of a variety of personal care products having as important properties one or more of body, gloss, moisturizing capability, viscosity enhancing and film forming attributes, and the like.

### BACKGROUND OF THE INVENTION

“Personal care products” is a term used to describe a wide variety of commercial offerings including cosmetics, shampoos and conditioners, deodorants and the like. Such products are typically formulated with polymeric materials to impart any of a number of properties of interest including gloss, sheen, and wettability and spreadability along surfaces such as skin and hair. Solubility of the polymer in water or alcohol is an important attribute to make it effective for use in such applications. Presently, there are many types of water- and alcohol-soluble polymers that are used in personal care or cosmetic applications (See “Cosmetic Applications”, Encyclopedia of Polymer Science & Engineering, Volume I, pages 18-30, Second Edition, John Wiley & Sons. See also “Hair Preparations”, Encyclopedia of Chemical Technology, Volume 12, pages 80-113, Third Edition, John Wiley & Sons). These polymers are either natural (biopolymers) or synthetic polymers. The natural polymers are also often modified to enhance or impart desirable properties. These polymers can be ionic or non-ionic. Uses for these polymers range from viscosity thickeners, emulsifiers, and protective colloids to moisturizers, film formers, binders, anti-static agents, and surfactants.

For such personal care formulations the polymers commonly used are polyvinyl acetate/crotonic acids, polyvinyl ether/maleic anhydrides, polyvinylpyrrolidinones and their copolymers, acrylamides, ethylene/acrylic acids, polyvinyl alcohols, and polyethylene glycols. Of particular note, polyethylene glycols are used widely for multiple purposes such as moisturizers, plasticizers, lubricants, and humectants, and when used in the form of ethoxylates are used as surfactants.

Concurrently there has long been an interest in the personal care product industry regarding the use of nylon-based materials for such formulations. Nylons are well known for their desirable properties such as strength, toughness, abrasion resistance, lubricity, and chemical resistance. They also have very high gloss from a physical appearance standpoint. Moreover polyamides are chemically similar to proteins and might be expected to assist with the wetting or spreading of formulations containing them along surfaces such as skin and hair.

However, in order for polyamides to be suitable for these uses they must effectively dissolve in water and alcohol, and this traditionally requires that such polymers must be made highly polar. A large number of functional groups such as amines, hydroxyls, sulfonic acids, and carboxylic acids and their salts are commonly used therefore to solubilize nylon polymers. One major drawback associated with this approach, is that it is not uncommon for the reactive functional groups such as acids and amines to chemically react with other acids and bases that they come in contact in their formulation or during end-use. This leads to undesirable changes in properties such as solubility. An extensive review of water-soluble polymers is covered in “Water Soluble Polymers”, Encyclopedia of Polymer Science & Engineering, Volume 17, pages 730-784, Second Edition, John Wiley & Sons (1989).

As a result of these limitations, polyamides have not been widely recognized as candidates for incorporation within personal care formulations. More specifically, polyamides derived from polyoxyethylene diamines and dicarboxylic acids have not been associated with personal care formulations. And yet there is a long-felt need to incorporate the functionalities of polyethylene glycols and the desirable properties of polyamides and make use of the good qualities of both polyethylene glycols and polyamides together. The resulting polyether amides would improve the compatibility of the polymer with proteins, thereby offering a tremendous advantage in personal care products.

It is an object of the present invention to provide water and alcohol soluble polyamides suitable for incorporation into personal care product formulations. It is a further object of the present invention to provide personal care products including such polyamides that exhibit good wettability and sheen. A feature of the present invention is the use of nylons for this purpose and without need to incorporate functional groups therein which may lead to undesirable side reactions. Another feature of products of the present invention is their retention of properties of interest including gloss, high moisture content and the like. It is an advantage of the present invention that the numerous beneficial attributes of polyamides including strength, lubricity and chemical resistance can be incorporated into personal care product lines. A further advantage of the present invention is its suitability for any number of personal care applications. These and other objects, features and advantages of the present invention will become better understood upon having reference to the description of the invention herein.

### SUMMARY OF THE INVENTION

There is disclosed and claimed herein a personal care formulation comprising (i) a water and alcohol soluble polyamide with a solubility at 23 C. of at least 0.5 weight percent and derived from the reaction of adipic acid and ether diamines with a molecular weight of 148 to 396 and represented by the general formulas

H₂N—R₁—O—R₂—O—R₁—NH₂

wherein R₁ and R₂ are either —CH₂—CH₂— or —CH₂—CH₂—CH₂—;

H₂N—R₁(—O—CH₂—CH₂—)ₓO—R₁—NH₂

wherein R₁ is either —CH₂—CH₂— or —CH₂—CH₂—CH₂— and X has an average value of 2 to 6; and mixtures thereof.; and

(ii) an effective amount of one or more of surfactants, dispersants, propellants, solvents, and/or other additives suitable to achieve the desired formulation.

Those of skill in the art will appreciate that R₁'s in the above formula are usually the same but can be different.

There is also disclosed and claimed herein processes for the manufacture of water and alcohol-soluble polyamide containing personal care products. The polyamide described above is formed as a liquid or in granular form. Appropriate substrate materials suitable for the application of interest are added thereto. Optional ingredients may be incorporated therein, again tailored to the application of interest. For example in various cosmetics applications any of one or more of a variety of colorants, dyes, pigments and the like may be added to impart a color of interest to the final product.

### DETAILED DESCRIPTION OF THE INVENTION

It should be considered that the term “personal care formulations” is intended to encompass a wide range of products, including without limitation hair sprays, cosmetics (such as makeup and lipsticks), deodorants, shampoos, conditioners, moisturizers, antiperspirants, and creams. The invention disclosed herein represents a significant advance in such personal care formulations, in that water soluble nylons are disclosed which are suitable for incorporation into these formulations, along with colorants, pH adjusters, thickeners, solvents, surfactants and the like as is necessary or desirable to make the product candidate in question.

For example, hair sprays rely on resinous materials as the primary setting agents. Hair sprays conventionally offer one or more of the following characteristics: hair holding properties; curl retention properties; little flake or powder on combing; rapid drying; nonstickiness; lustrous effect; removal on shampooing; nontoxicity; and resistance to microbial contamination. Accordingly, in addition to the resinous material any number of ingredients are added to accomplish these purposes and including plasticizers or other film modifying additives, solvent systems, and propellants. Those having skill in the art to which this invention pertains (and in particular to the product candidate in question, here hair sprays) will readily appreciate that the instant personal care formulations include the water soluble nylon material claimed, and together with the additional ingredients as are suitable for hair spray manufacture and use. Furthermore such persons of relevant skill will use “effective amounts” of these various ingredients, meaning an amount suited to achieve the desired effect, as is understood within the field. Typically the amounts of water soluble nylon useful in hairsprays may range from 1 to 25 weight percent, and typically preferably in the range of 2 to 15 weight percent for hairspray formulations, with the balance of the various ingredients as above providing the remaining weight percent of the formulation. Similar considerations apply when incorporating the water soluble nylon materials herein into other product lines, for example cosmetics or deodorants. For example as a thickener in colorant formulations, about 3 weight percent (and in a range of about 1 to 5 weight percent) of water soluble nylon may be typically used.

Copolyamides of the above polyamide with other polyamide-forming comonomers can also be used herein. These other nylon forming comonomers may be incorporated into the compositions and products described herein provided these comonomers do not adversely affect the water solubility of the resulting polyamide. These added comonomers may include other polyamide forming comonomers such as lactams, polyether diamines, polyether diacids, alkylene diamines, and alkylene dicarboxylic acids. The solubility in water of these nylons is influenced not only by the amount of the polyetherdiamines and the nature of the dicarboxylic acids but the molecular weight as well. Polyethylene glycol diamines and diacids are of particular interest as a polyamide-forming comonomer with the polyamides described above. Moreover, other personal care formulations of note include those wherein the above described water and alcohol soluble polyamide is a copolymer with caprolactam and polyamides derived from hexamethylene diamine or 2-methylpentamethylene diamine and adipic acid or mixtures thereof.

Additives such as heat and UV stabilizers, anti-oxidants, plasticizers, lubricants, and catalyst may be used if desired to enhance the properties of the polymer or aid the polymerization process. Those having skill in the art to which this invention pertains will readily appreciate how much and in what manner these additives may be incorporated.

The water-soluble personal care products disclosed herein may take the form of any number of products, and can be broadly be classified into “liquid” based products and “solid” based products. Liquid products include without limitation shampoos, conditioners, moisturizers, deodorants, antiperspirants, and creams. Solid products include without limitation makeup materials and lipsticks. In these and related applications, the polyamides are important contributors to body, gloss, as a binder, viscosity thickening agent, film forming agent, and moisturizing component among many other desirable properties. They also offer flexibility in choice of solvent.

### EXAMPLES

Preparation of the Nylon Resins

The nylon polymerization was carried out using standard nylon polymerization process that is well-known in the art (See Kohan, M. I., “Nylon Plastics Handbook” Hansen/Gardner Publications, Inc. [1995] pages 17-20 & 34-45). As is well-known in the art, the stoichiometry of the ingredients was determined and controlled using pH measurements. The molecular weight during polymerization, as indicated by relative viscosity (RV), was controlled by controlling pH, use of atmospheric, nitrogen, or vacuum finishing after pressure reduction. Usually, the molten polymer was quenched in water and then cut into pellets. However, because these nylons are water-soluble the molten polymer was either allowed to cool under ambient conditions or dropped onto a bed of ground dry ice for cooling.

Testing

The relative viscosity in formic acid (RV) of an 8.4% solution was determined at 25 C. using a Brookfield Viscometer.

The solubility in room temperature water (23 C.) at 10% concentration was determined by mixing 10 weight percent of the polymer with 90 weight percent demineralized water and stirring at room temperature. The solution was allowed to sit at room temperature and the solution was observed for any sign of precipitation.

### Comparative Example A

In a beaker provided with a stirrer, 300 ml. of demineralized water and 222.0 g of triethyleneglycol diamine (H₂N—CH₂—CH₂—O—CH₂—CH₂—O—CH₂—CH₂—NH₂) were mixed and heated to 60-70 C. with stirring. To the mixture was added slowly 345.0 g dodecanedioic acid. An additional 200 ml of de-mineralized water was added. When all the dodecanedioic acid was dissolved the pH was adjusted to 7.15 by addition of 4.1 g of triethyleneglycol diamine (TEGD). The solution was then introduced into a 3,785 ml autoclave where the solution was heated slowly until the pressure in the autoclave reached 250 psig. At this point, steam was slowly vented while heating was continued. When the batch temperature reached 225 C., the steam venting was increased so as to lower the pressure to atmospheric pressure in 45 minutes but at a rate such that the batch temperature would continue to increase as it was being concentrated. The polymer was then subjected to 21.0 ″ of vacuum for 60 minutes. At the end of 60 minutes the batch temperature was 270 C. The autoclave was then pressured with nitrogen and forced out of the autoclave and into a pan. The polymer was allowed to cool to room temperature. The polymer had an RV of 15.7.

Using the same procedure (but with minor variations in temperature, vacuum and hold time as appropriate by those of skill in the art, to obtain the desired molecular weight) as Comparative Example A, Comparative Examples B and C were prepared using the appropriate ingredients. Results are shown below.

| SAMPLE | COMPOSITION | CATALYST | RV | SOLUBILITY |
|---|---|---|---|---|
| Comparative | TEGD, 9 | None | 14.9 | Insoluble |
| Example C | | | | |
| Comparative | TEGD,10 | None | 13.3 | Insoluble |
| Example B | | | | |
| Comparative | TEGD,12 | None | 15.7 | Insoluble |
| Example A | | | | |

### Example 1

In a beaker provided with a stirrer, 300 ml. of de-mineralized water and 444.0 g of TEGD were mixed and heated to 60-70 C. with stirring. To the mixture was added slowly 438.0 g of adipic acid. An additional 100 ml of de-mineralized water was added. When all the adipic acid was dissolved the pH was adjusted to 7.25 by addition of 7.2 g of TEGD. The solution was then introduced into a 3,785 ml autoclave where the solution was heated slowly until the pressure in the autoclave reached 250 psig. At this point, steam was slowly vented while heating was continued. When the batch temperature reached 225 C., the steam venting was increased so as to lower the pressure to atmospheric pressure in 45 minutes but at rate such that the batch temperature would continue to increase as it was being concentrated. The polymer was then subjected to 19.5″ of vacuum for 60 minutes. At the end of 60 minutes the batch temperature was 270 C. The autoclave was then pressured with nitrogen and forced out of the autoclave and into a pan. The polymer was allowed to cool to room temperature. The polymer had an RV of 12.9.

### Example 2

In a beaker provided with a stirrer, 1997.0 g of de-mineralized water and 740.0 g of TEGD were mixed with stirring. To the mixture was added slowly 730.0 g of adipic acid. When all the adipic acid was dissolved 0.37 g of sodium hypophosphite monohydrate (SHP monohydrate) was added. The pH of the salt solution was 7.10. An 830.0 g portion of the salt was then introduced into a 3,785 ml autoclave where the solution was heated slowly until the pressure in the autoclave reached 250 psig. At this point, steam was slowly vented while heating was continued. When the batch temperature reached 225 C., the steam venting was increased so as to lower the pressure to atmospheric pressure in 45 minutes but at a rate such that the batch temperature would continue to increase as it was being concentrated. The polymer was then held at atmospheric conditions for 20 minutes. At the end of 20 minutes the batch temperature was 255 C. The autoclave was then pressured with nitrogen and forced out of the autoclave and into a pan with ground dry ice. The polymer had an RV of 14.0.

Example 3 and Example 4 were prepared under the same procedure as Example 2 with the exception that vacuum was used for the finishing step. The results are shown below.

| SAMPLE | COMPOSITION | CATALYST (1) | RV | SOLUBILITY |
|---|---|---|---|---|
| Example 1 | TEGD,6 | None | 12.9 | Soluble |
| Example 2 | TEGD,6 | 210 ppm | 14.0 | Soluble |
| Example 3 | TEGD,6 | 349 ppm | 20.5 | Soluble |
| Example 4 | TEGD,6 | 210 ppm | 22.8 | Soluble |

(1) Sodium hypophosphite monohydrate.

Examples 1 to 4 and Comparative Examples A, B, and C demonstrate that the incorporation of ether amine segments in the polymer alone is not sufficient to achieve water solubility. The proper selection of the dicarboxylic acid structure is necessary to obtain water soluble nylons.

### Example 5

In a beaker provided with a stirrer, 300 ml of de-mineralized water and 278.2 g of TEGD were mixed and heated to 60-70 C. with stirring. To the mixture was added slowly 274.5 g of adipic acid. When the adipic acid has dissolved, 269.0 g of caprolactam solution with an 81.86 weight percent concentration was added. The pH was then adjusted to 7.35 by addition of 4.1 g of TEGD. The solution was then introduced into a 3,785 ml autoclave where the solution was heated slowly until the pressure in the autoclave reached 250 psig. At this point, steam was slowly vented while heating was continued. When the batch temperature reached 225 C., the steam venting was increased so as to lower the pressure to atmospheric pressure in 45 minutes but at a rate such that the batch temperature would continue to increase as it was being concentrated. The polymer was then subjected to 22.0″ to 22.5″ of vacuum for 60 minutes. At the end of 60 minutes the batch temperature was 268 C. The autoclave was then pressured with nitrogen and forced out of the autoclave and into a pan. The polymer was allowed to cool to room temperature. The polymer had an RV of 17.7. The results are shown below.

| | COMPO- | MOLE | CATALYST | | |
|---|---|---|---|---|---|
| SAMPLE | SITION | RATIO | (1) | RV | SOLUBILITY |
| Example 5 | TEGD,6/6 | 50/50 | None | 17.7 | Soluble |
| Example 6 | TEGD,6/6 | 50/50 | 349 ppm | 25.6 | Soluble (2) |
| Example 7 | TEGD,6/6 | 70/30 | None | 15.3 | Soluble |
| Example 8 | TEGD,6/6 | 80/20 | None | 14.1 | Soluble |
| Comparative | TEGD,6/6 | 40/60 | None | 19.0 | Insoluble |
| Example D | | | | | |

(1) Sodium hypophosphite monohydrate
(2) Soluble but went to solution much slower than Example 5.

Examples 5, 6, 7, 8, and Comparative Example D illustrate that the ratio of comonomers affect the solubility of the copolymers in water. Example 5 and Example 6 also demonstrate that the RV (molecular weight) of the polymer also affects the rate of solution. The higher molecular weight results in slower dissolution rate.

### Examples 9 to 11 and Comparative Examples E, F, G and H

Using the same procedure as in previous examples and controlling RV as previously described herein, various copolymers with nylon 66, 46, and 2-methylpentamethylenediamine,6 were prepared. The results are shown below.

| | COMPO- | MOLE | CATALYST | | SOLU- |
|---|---|---|---|---|---|
| SAMPLE | SITION (1) | RATIO | (2) | RV | BILITY |
| Example 9 | TEGD,6/6,6 | 90/10 | None | 14.1 | Soluble |
| Comparative | TEGD,6/6,6 | 80/20 | None | 15.5 | Insoluble |
| Example E | | | | | |
| Comparative | TEGD,6/6,6 | 70/30 | 152 ppm | 16.5 | Insoluble |
| Example F | | | | | |
| Example 10 | TEGD, | 70/30 | None | 15.1 | Soluble |
| | 6/2MPMD,6 | | | | |
| Comparative | TEGD, | 65/35 | None | 17.7 | Insoluble |
| Example G | 6/2MPMD,6 | | | | |
| Example 11 | TEGD,6/4,6 | 70/30 | None | 9.4 | Soluble |
| Comparative | TEGD,6/4,6 | 50/50 | None | 10.5 | Insoluble |
| Example H | | | | | |

(1) 2MPMD stands for 2-methylpentamethylenediamine
(2) Sodium hypophosphite monohydrate

Examples 9, 10, 11, and Comparative Examples E, F, G, and H illustrate again that the solubility in water of copolymers is dependent on the type and amount of comonomer used.

### Example 12

In a beaker provided with a stirrer, 500 ml of demineralized water and 264.0 g of 1,2-bis(gamma-aminopropoxy)ethane (H₂N—CH₂—CH₂—CH₂—O—CH₂—CH₂—O—CH₂-CH₂—CH₂—NH₂ ) were mixed and heated to 60-70 C. with stirring. To the mixture was added slowly 219.0 g of adipic acid. When the adipic acid has dissolved the pH was adjusted to 7.12 by adding 26.0 g of 1,2-bis(gamma-aminopropoxy)ethane (BGAE) and 5.0 g of adipic acid. Those having skill in the art to which the invention pertains will readily appreciate that different grades of BGAE (and as described later, POE-DPA 220) are available, and these have differing levels of monoamines and triamines associated with them. However these byproducts have minor effects in adjusting the pH level so that the pH of interest is readily attained. This may have an effect on the polymerization process, and some adjustments to this process may be necessary to achieve the desirable molecular weight, again as is well appreciated by the person of skill. The solution was then introduced into a 3,785 ml autoclave where the solution was heated slowly until the pressure in the autoclave reached 250 psig. At this point, steam was slowly vented while heating was continued. When the batch temperature reached 225 C., the steam venting was increased so as to lower the pressure to atmospheric pressure in 45 minutes but at a rate such that the batch temperature would continue to increase as it was being concentrated. The polymer was then subjected to 21-22″ of vacuum for 60 minutes. At the end of 60 minutes the batch temperature was 258 C. The autoclave was then pressured with nitrogen and forced out of the autoclave and into a pan. The polymer was allowed to cool to room temperature. The polymer had an RV of 7.7.

### Example 13

In a beaker provided with a stirrer, 500 ml of de-mineralized water, 246.4 g of BGAE, and 82.0 g of caprolactam solution with an 82.68 weight percent concentration were mixed and heated to 60-70 C. with stirring. To the mixture was added slowly 204.4 g of adipic acid. When the adipic acid has dissolved the pH was adjusted to 7.09 by adding 19.5 g of BGAE. The solution was then introduced into a 3,785 ml autoclave where the solution was heated slowly until the pressure in the autoclave reached 250 psig. At this point, steam was slowly vented while heating was continued. When the batch temperature reached 225 C., the steam venting was increased so as to lower the pressure to atmospheric pressure in 45 minutes but at a rate such that the batch temperature would continue to increase as it was being concentrated. The polymer was then subjected to 21″ of vacuum for 60 minutes. At the end of 60 minutes the batch temperature was 264 C. The autoclave was then pressured with nitrogen and forced out of the autoclave and into a pan. The polymer was allowed to cool to room temperature. The polymer had an RV of 8.7.

### Comparative Example I

In a beaker provided with a stirrer, 500 ml of de-mineralized water, 211.2 g of BGAE, and 164.0 g of caprolactam solution with an 82.68 weight percent concentration were mixed and heated to 60-70 C. with stirring. To the mixture was added slowly 175.2 g of adipic acid. When the adipic acid has dissolved the pH was adjusted to 7.15 by adding 12.0 g of BGAE. The solution was then introduced into a 3,785 ml autoclave where the solution was heated slowly until the pressure in the autoclave reached 250 psig. At this point, steam was slowly vented while heating was continued. When the batch temperature reached 225 C., the steam venting was increased so as to lower the pressure to atmospheric pressure in 45 minutes but at a, rate such that the batch temperature would continue to increase as it was being concentrated. The polymer was then subjected to 18-19″ of vacuum for 60 minutes. At the end of 60 minutes the batch temperature was 264 C. The autoclave was then pressured with nitrogen and forced out of the autoclave and into a pan. The polymer was allowed to cool to room temperature. The polymer had an RV of 10.7. The results are shown below.

| | | MOLE | | |
|---|---|---|---|---|
| SAMPLE | COMPOSITION (1) | RATIO | RV | SOLUBILITY |
| Example 12 | BGAE,6 | | 7.7 | Soluble |
| Example 13 | BGAE,6/6 | 70/30 | 8.7 | Soluble |
| Comparative | BGAE,6/6 | 50/50 | 10.7 | Insoluble |
| Example I | | | | |

(1) BGAE is an acronym for 1,2-bis(gamma-aminopropoxy)ethane

Examples 12, 13, and Comparative Example I show that replacement of TEGD with BGAE also affords a water-soluble polyamide. Furthermore, copolymers of BGAE,6 behaves similarly with the copolymers of TEGD,6.

### Example 14

In a beaker provided with a stirrer, 300 ml of de-mineralized water and 176.0 g of POE-DPA220 were mixed and heated to 60-70 C. with stirring. This diprimary amine has the following structure (H₂N—CH₂—CH₂—CH₂-[polyoxyethylene]-CH₂—CH₂-CH₂—NH₂) where the polyoxyethylene unit is (O—CH₂—CH₂—O—CH₂—CH₂—O) and has a molecular weight of 220. To the mixture was added slowly 116.8 g of adipic acid. The pH of the solution was 6.99. To the solution was added 0.074 g of sodium hypophosphite monohydrate. The salt solution was then introduced into a 3,785 ml autoclave where the solution was heated slowly until the pressure in the autoclave reached 250 psig. At this point, steam was slowly vented while heating was continued. When the batch temperature reached 225 C., the steam venting was increased so as to lower the pressure to atmospheric pressure in 45 minutes but at a rate such that the batch temperature would continue to increase as it was being concentrated. The polymer was then held at atmospheric pressure for 20 minutes. At the end of 20 minutes the batch temperature was 249 C. The autoclave was then pressured with nitrogen and forced out of the autoclave and into a pan of dry ice. The polymer had an RV of 7.8 and was soluble in water at room temperature.

### Comparative Example J

In a beaker provided with a stirrer, 200 ml of de-mineralized water and 88.0 g of POE-DPA220 were mixed and heated to 60-70 C. with stirring. To the mixture was added slowly 58.4 g of adipic acid. The pH of the solution was adjusted to 6.72 by addition of 5.0 g of POE-DPA220. To the solution were added 117.6 g of a caprolactam solution with a concentration of 74.69 weight percent, 186.8 g of nylon 6,6 salt with a concentration of 31.35 weight percent, and 0.88 g of sodium hypophosphite monohydrate. The salt solution was then introduced into a 3,785 ml autoclave where the solution was heated slowly until the pressure in the autoclave reached 250 psig. At this point, steam was slowly vented while heating was continued. When the batch temperature reached 225 C., the steam venting was increased so as to lower the pressure to atmospheric pressure in 45 minutes but at a rate such that the batch temperature would continue to increase as it was being concentrated. The polymer was then held at atmospheric pressure for 18 minutes. At the end of 18 minutes the batch temperature was 260 C. The autoclave was then pressured with nitrogen and forced out of the autoclave into a pan of dry ice. The polymer had an RV of 12.5.

Using the same procedure as Comparative Example J, and controlling RV as previously described herein, Comparative Examples K and L were prepared using POE-DPA514 (molecular weight of 514) and POE-DPA1114 (molecular weight of 1114). The results are shown below.

| | | SALT | | | |
|---|---|---|---|---|---|
| | COMPO- | WT. | | | SOLU- |
| SAMPLE | SITION (1) | RATIO | CATALYST | RV | BILITY |
| Comparative | POE- | 50/20/30 | 0.30 wt. % | 12.5 | Insoluble |
| Example J | DPA220, | | | | |
| | 6/6,6/6 | | | | |
| Comparative | POE- | 50/20/30 | 0.30 wt. % | 15.9 | Insoluble |
| Example K | DPA514, | | | | |
| | 6/6,6/6 | | | | |
| Comparative | POE- | 50/20/30 | 0.29 wt. % | 16.4 | Insoluble |
| Example L | DPA1114, | | | | |
| | 6/6,6/6 | | | | |

Comparative Examples J, K, and L are polymers containing polyether amines and are described in U.S. Pat. No. 4,323,639 and U.S. Pat. No. 5,688,632 as water-soluble. These comparative examples show that the water-soluble nylon described in the U.S. Pat. No. 4,323,639 and U.S. Pat. No. 5,688,632 are not water soluble and are not useful for the purposes of this invention.

It will be readily apparent that any number of variations and modifications to the subject matter disclosed and claimed herein can be made, and are contemplated as within the scope and purview of the invention herein.

In the claims

1. A personal care formulation comprising (i) a water and alcohol soluble polyamide with a solubility at 23 C. of at least 0.5 weight percent and derived from the reaction of adipic acid and ether diamines with a molecular weight of 148 to 396 and represented by the general formulas
H₂N—R₁—O—R₂—O—R₁—NH₂
wherein R₁ and R₂ are either —CH₂—CH₂— or —CH₂—CH₂—CH₂—;
H₂N—R₁(—O—CH₂—CH₂—)ₓO—R₁—NH₂
wherein R₁ is either —CH₂—CH₂— or —CH₂—CH₂—CH₂— and X has an average value of 2 to 6; and mixtures thereof; and
(ii) an effective amount of one or more of surfactants, dispersants, propellants, solvents, and/or other additives suitable to achieve the desired formulation.

2. The personal care formulation of claim 1 further comprising copolyamides of said water and alcohol soluble polyamide and one or more polyamide-forming comonomers.

3. The personal care formulation of claim 2 wherein said polyamide-forming comonomer is polyethylene glycol diamine or polyethylene glycol diacids or mixtures thereof.

4. The personal care formulation in claim 1 where said water and alcohol soluble polyamide is a copolymer with caprolactam and polyamides derived from hexamethylene diamine or 2-methylpentamethylene diamine and adipic acid or mixtures thereof.

5. The personal care formulation of claim 1 in the form of a liquid.

6. The personal care formulation of claim 1 wherein said liquid is selected from the group consisting of shampoos, conditioners, moisturizers, deodorants, antiperspirants, and creams.

7. The personal care formulation of claim 1 in the form of a solid.

8. The personal care formulation of claim 7 wherein said solid is selected from the group consisting of makeup materials and lipsticks.

9. The personal care formulation of claim 1 in the form of a spray.

10. The personal care formulation of claim 9 wherein said spray is a hair spray.

11. A process for the manufacture of water- and alcohol-soluble polyamide containing personal care products comprising:
(i) Forming a water-and alcohol-soluble polyamide as a liquid or in granular form, with a solubility at 23 C. of at least 0.5 weight percent and derived from the reaction of adipic acid and ether diamines with a molecular weight of 148 to 396 and represented by the general formulas
H₂N—R₁—O—R₂—O—R₁—NH₂
wherein R₁ and R₂ are either —CH₂—CH₂— or —CH₂—CH₂—CH₂—;
H₂N—R₁(—O—CH₂—CH₂—)ₓO—R₁—NH₂
wherein R₁ is either —CH₂—CH₂— or —CH₂—CH₂—CH₂— and X has an average value of 2 to 6; and mixtures thereof;
(ii) Adding thereto a substrate material suitable for the application of interest; and
(iii) Optionally adding thereto one or more of colorants, dyes, or pigments sufficient to impart thereto a color of interest.

12. The process of claim 1 1 wherein said water- and alcohol-soluble polyamide is a liquid and said substrate materials are selected according to any of applications selected from the group consisting of shampoos, conditioners, moisturizers, deodorants, antiperspirants, and creams.

13. The process of claim 11 wherein said water- and alcohol-soluble polyamide is granular and said substrate materials are selected according to any of applications selected from the group consisting of makeup and lipsticks.
